(19) United States
(12) Patent Application Publication
(10) Pub. No.: US 2021/0208075 A1
(43) Pub. Date: Jul. 8, 2021

(54) AUTOFLUORESCENCE QUENCHING ASSAY AND DEVICE

(71) Applicant: Sumitomo Chemical Company, Limited, Tokyo (JP)

(72) Inventors: Matthew Roberts, Godmanchester (GB); May Wheeler, Godmanchester (GB)

(73) Assignee: Sumitomo Chemical Company, Limited, Tokyo (JP)

(21) Appl. No.: 16/650,741

(22) PCT Filed: Sep. 13, 2018
(86) PCT No.: PCT/GB2018/052607

§ 371 Date: Mar. 25, 2020

(30) Foreign Application Priority Data
Sep. 29, 2017 (GB) 1715774.4

(51) Int. Cl. G01N 21/64; G01N 33/53; G01N 33/542; G01N 33/543; G01N 33/558

(52) U.S. Cl. CPC G01N 21/6486; G01N 21/6428; G01N 33/5302; G01N 33/542; G01N 2021/6491; G01N 33/54393; G01N 33/558; G01N 2021/6432; G01N 33/54386

(57) ABSTRACT

A fluorescence quenching immunoassay method includes determining the presence or concentration of an analyte in a liquid sample in dependence upon quenching of an autofluorescence signal of a substrate. Devices for conducting the fluorescence quenching immunoassay method are also described.

### FIELD

The present invention relates to a method and device for fluorescence quenching assays.

### BACKGROUND

Biological testing for the presence and/or concentration of an analyte may be conducted for a variety of reasons including, amongst other applications, preliminary diagnosis, screening samples for presence of controlled substances and management of long term health conditions.

Lateral flow devices (also known as “lateral flow immunoassays”) are one variety of biological testing. Lateral flow devices may be used to test a liquid sample, such as saliva, blood or urine, for the presence of an analyte. Examples of lateral flow devices include home pregnancy tests, home ovulation tests, tests for other hormones, tests for specific pathogens and tests for specific drugs. For example, EP 0 291 194 A1 describes a lateral flow device for performing a pregnancy test.

In a typical lateral flow testing strip, a liquid sample is introduced at one end of a porous strip, and the liquid sample is then drawn along the strip by capillary action (or “wicking”). A portion of the lateral flow strip is pre-treated with labelling particles which are activated with a reagent which binds to the analyte to form a complex, if the analyte is present in the sample. The bound complexes and also unreacted labelling particles continue to propagate along the strip before reaching a testing region which is pre-treated with an immobilised binding reagent which binds bound complexes of analyte and labelling particles and does not bind unreacted labelling particles. The labelling particles have a distinctive colour, or other detectable optical or non-optical property, and the development of a concentration of labelling particles in the test region or regions provides an observable indication that the analyte has been detected. Lateral flow test strips may be based on, for example, colorimetric labelling using gold or latex nanoparticles, fluorescent marker molecules or magnetic labelling particles. In other lateral flow test strips, the concentration of analyte is measured based on using labelling particles/substances which quench of the fluorescence of fluorescent dyes, tags or markers immobilised within a test portion of the lateral flow strip.

Another variety of biological testing involves assays conducted in liquids held in a container such as a vial, a PCR well/plate, a cuvette or a microfluidic cell. Liquid assays may be measured based on colorimetry, fluorescence or fluorescence quenching. An advantage of some liquid based assays is that they may allow tests to be conducted using very small (e.g. picolitre) volumes.

Chen et al.: “Antigen detection based on background fluorescence quenching immunochromatographic assay”, Analytica Chimica Acta, Volume 841, 2 Sep. 2014, Pages 44 to 50, describes a quenching complex which is introduced to quench the fluorescence of a fluorescein dye coated on the entire nitrocellulose membrane of a nitrocellulose lateral flow strip. A liquid sample thought to contain analyte can be added to a sample pad, and mixed with a quenching complex to form an analyte-quenching complex as the liquid sample flows through a conjugate pad. The analyte-quenching complex binds to a test line and acts to reduce the total fluorescent signal. This reduction in signal will correlate with the analyte concentration. This device requires the entire nitrocellulose membrane to be coated with a fluorescent dye, and furthermore, at low analyte concentrations, sensitivity may be limited by the autofluorescence of the nitrocellulose membrane.

### SUMMARY

According to a first aspect, there is provided a fluorescence quenching immunoassay method, including determining the presence or concentration of an analyte in a liquid sample in dependence upon quenching of an autofluorescence signal of a substrate.

The substrate may be formed from a material which is inherently fluorescent.

The substrate does not contain or support any added fluorophores or phosphorescing substances. The substrate may not have been treated with any fluorescent dyes, fluorescent tags, or fluorescent molecules.

The substrate may be porous. The substrate may be formed from a fibrous material. The fibrous material may take the form of a mat, a felt or a cloth of fibres. The substrate may be formed of natural fibres. The substrate may be formed of synthetic organic fibres.

The substrate may be formed from fibres comprising one or more of cellulose, nitrocellulose, lignin, collagen, cotton and silk. The substrate may be formed from paper.

The method may be conducted using a lateral flow immunoassay device.

The method may also include determining the presence or concentration of the analyte in dependence upon a combination of quenching of the autofluorescence signal of the substrate and an absorbance of a quenching substance which causes quenching of the substrate autofluorescence signal.

The substrate may include at least one test region treated with an immobilised binding reagent for selectively binding an analyte-quenching complex. The method may also include exposing the liquid sample to a quenching substance for selectively binding to the analyte to form an analyte-quenching complex. The method may also include applying the exposed liquid sample to the substrate. The method may also include determining, for each test region, the presence or concentration of the analyte in dependence upon a decrease in a corresponding autofluorescence signal.

The quenching substance may include a quenching label which quenches the substrate autofluorescence, the quenching label bound to an antibody or an antigen. The immobilised binding agent may include an antibody or an antigen. The quenching label may quench the substrate autofluorescence by energy transfer, by electron transfer and/or by absorbing light at one or more excitation wavelengths of the substrate material.

The quenching substance may include gold nanoparticles.

The substrate autofluorescence may be excited by one or more excitation wavelengths, and the quenching substance may absorb within a first wavelength range which overlaps one or more excitation wavelengths. The presence or concentration of the analyte may be determined, for each test region, in dependence upon a combination of the change in the autofluorescence signal from the test region in response to illumination with light of the first wavelength range, and a change in absorbance of the test region within the first wavelength range.

The method may be conducted using a lateral flow immunoassay device which includes a substrate in the form of a porous strip, the porous strip comprising the at least one test region, and a porous conjugate pad arranged to permit fluid communication with the porous strip, the conjugate pad comprising the quenching substance.

According to a second aspect, there is provided a device configured to carry out the method.

According to a third aspect, there is provided a kit adapted to carry out the method.

According to a fourth aspect, there is provided a fluorescence quenching immunoassay device configured to determine the presence or concentration of an analyte in a liquid sample. The device includes a substrate which exhibits autofluorescence. The device also includes one or more photodetectors configured to detect an autofluorescence signal of the substrate. The device also includes a controller configured to determine the presence or concentration of the analyte in dependence upon quenching of the autofluorescence signal of the substrate.

The substrate may be formed from a material which is inherently fluorescent.

The substrate does not contain or support any added fluorophores or phosphorescing substances. The substrate may not have been treated with any fluorescent dyes, fluorescent tags, or fluorescent molecules.

The substrate may be porous. The substrate may be formed from a fibrous material. The fibrous material may take the form of a mat, a felt or a cloth of fibres. The substrate may be formed of natural fibres. The substrate may be formed of synthetic organic fibres.

The substrate may be formed from fibres comprising one or more of cellulose, nitrocellulose, lignin, collagen, cotton and silk. The substrate may be formed from paper.

The device may take the form of a lateral flow immunoassay device.

The one or more photodetectors may include at least one first photodetector configured to detect an autofluorescence signal of the substrate. The one or more photodetectors may include at least one second photodetector configured to detect an absorbance of a quenching substance which causes quenching of the substrate autofluorescence signal. The controller may be further configured to determine the presence or concentration of the analyte in dependence upon a combination of quenching of the autofluorescence signal of the substrate and the absorbance of the quenching substance.

The device may include a porous sample receiving pad for receiving the liquid sample. The device may also include a porous conjugate pad arranged to permit fluid communication with the sample receiving pad. The conjugate pad may include a quenching substance for selectively binding to the analyte to form an analyte-quenching complex. The substrate may take the form of a porous test pad arranged to permit fluid communication with the conjugate pad. The test pad may include at least one test region treated with an immobilised binding agent for selectively binding analyte-quenching complexes. The test pad may be formed of a material which exhibits autofluorescence. The device may also include a wick pad arranged to permit fluid communication with the test pad. Each photodetector may be configured to detect an autofluorescence signal of a corresponding test region. The controller may be configured to determine, for each test region, the presence or concentration of the analyte in dependence upon a decrease in a corresponding autofluorescence signal. The liquid sample applied to the sample receiving pad may be drawn towards the wick pad via the conjugate pad and the test pad by a wicking mechanism.

The device may also include one or more light emitters configured to excite autofluorescence of the substrate.

The quenching substance may include a quenching label which quenches the substrate autofluorescence. The quenching label may be bound to an antibody or an antigen. The immobilised binding agent may include an antibody or an antigen. The quenching label may quench the substrate autofluorescence by energy transfer, by electron transfer and/or by absorbing light at one or more excitation wavelengths of the substrate material.

The quenching substance may include gold nanoparticles.

The one or more light detectors may include at least first and second photodetectors. The substrate autofluorescence may be excited by one or more excitation wavelengths. The quenching substance may absorb light within a first wavelength range which overlaps one or more excitation wavelengths. The controller may be configured to determine, for each test region, the presence or concentration of the analyte in dependence upon a combination of a change in the autofluorescence signal from the test region in response to illumination with light of the first wavelength range, measured using the first photodetector, and a change in absorbance of the test region within the first wavelength range, measured using the second photodetector.

The first and second photodetectors may be interdigitated.

### BRIEF DESCRIPTION OF THE DRAWINGS

Certain embodiments of the present disclosure will now be described, by way of example, with reference to the accompanying drawings in which:

FIG. 1 illustrates a fluorescence quenching immunoassay method;

FIG. 2 is a process flow diagram for a fluorescence quenching immunoassay method;

FIG. 3 shows a spectrum of autofluorescence for substrates containing nitrocellulose when illuminated using ultraviolet (UV) light;

FIG. 4 shows a spectrum of autofluorescence for substrates containing nitrocellulose when illuminated using blue light;

FIG. 5 shows fluorescence spectra for a fluorescent protein R-PE on a substrate made of nitrocellulose;

FIG. 6 shows variations in peak fluorescence intensity as a function of the area density of a fluorescent protein R-PE tag;

FIG. 7 shows the photoluminescence quantum yield (PLQY) measured for nitrocellulose;

FIG. 8 illustrates a first method of exposing a liquid sample to a quenching substance;

FIG. 9 illustrates a second method of exposing a liquid sample to a quenching substance;

FIG. 10 illustrates a third method of exposing a liquid sample to a quenching substance;

FIG. 11 illustrates a fourth method of exposing a liquid sample to a quenching substance;

FIGS. 12A and 12B illustrate a method of conducting a fluorescence quenching immunoassay using a lateral flow test strip;

FIG. 13 illustrates a first fluorescence quenching immunoassay device;

FIG. 14 illustrates a second fluorescence quenching immunoassay device;

FIG. 15 illustrates a third fluorescence quenching immunoassay device;

FIG. 16 illustrates a combined fluorescence quenching and absorbance immunoassay device;

FIG. 17 illustrates estimating an analyte concentration based on a combination of fluorescence quenching by a quenching label and absorbance of the quenching label; and

FIG. 18 illustrates a self-contained, single use lateral flow testing device incorporating a fluorescence quenching immunoassay device.

### DETAILED DESCRIPTION OF CERTAIN EMBODIMENTS

Fluorescence quenching assays have been proposed based on quenching the fluorescence of a fluorescent dye, tag, molecule or other similar fluorescent or phosphorescent marker. Such specifically added fluorescent markers, also sometimes referred to as fluorophores, are coated onto a substrate used to conduct the assay. This adds complexity and expense to production of the assay. Furthermore, the substrate is often made from a material which exhibits autofluorescence, in other words the substrate material itself may be inherently fluorescent. The autofluorescence of the substrate has been regarded as a source of error which must be minimised or circumvented. For example, by using a phosphorescent dye for time-gating to remove autofluorescence, or by using a dye with a large Stoke's shift.

In the present specification, methods and devices shall be explained which permit a fluorescence quenching assay to be conducted without the need to add any fluorescent dye, tag, molecule or other similar fluorescent or phosphorescent marker. Instead, the present specification is concerned with fluorescence quenching immunoassay methods and devices which are based on quenching the autofluorescence of the substrate material.

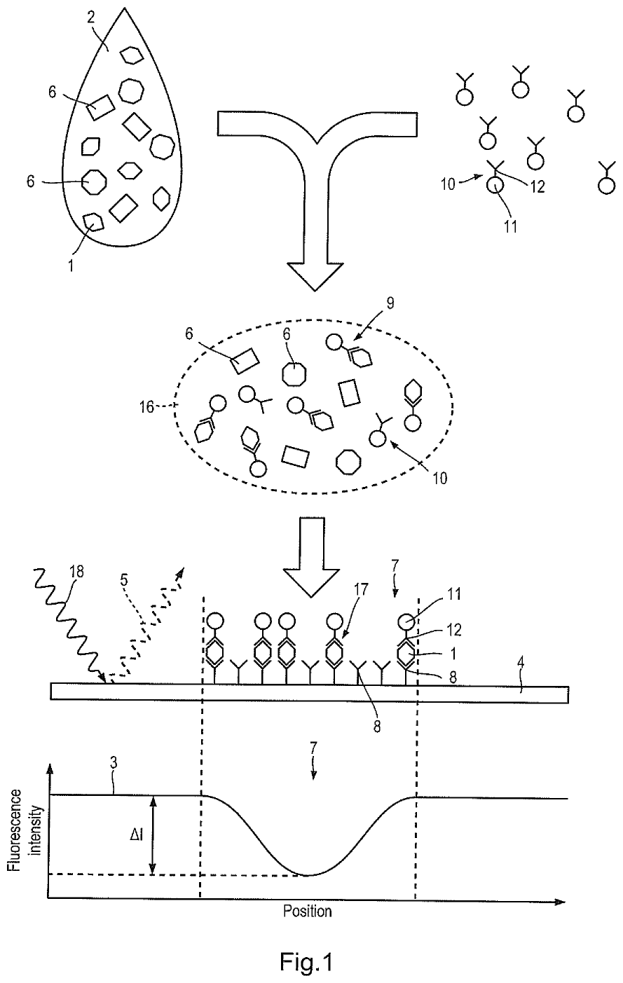

Referring to FIG. 1, a fluorescence quenching immunoassay method is illustrated.

The fluorescence quenching immunoassay method includes determining the presence or concentration of an analyte 1 in a liquid sample 2, in dependence upon quenching of an autofluorescence signal 3 of a substrate 4, in the form of autofluorescence light 5. The liquid sample 2 may include molecules/substances 6 other than the analyte 1, and the fluorescence quenching immunoassay method is specific to the presence or concentration of the analyte 1 of interest.

The substrate 4 is formed from a material which is inherently fluorescent. In other words, the substrate 4 does not include and/or has not been treated with any fluorophores, or any phosphorescing substances, to make the substrate 4 fluoresce. For example, the substrate 4 is not treated with any fluorescent or phosphorescent dyes, fluorescent or phosphorescent tags, or fluorescent or phosphorescent molecules.

The substrate 4 is porous, and may be formed from a fibrous material. Fibrous material forming the substrate 4 may take the form of a mat, a felt or a cloth of fibres. The substrate 4 may be formed of natural fibres or synthetic organic fibres such as, for example, from fibres comprising one or more of cellulose, nitrocellulose, lignin, collagen, cotton and silk. Preferably the substrate 4 is formed of nitrocellulose fibres. However, in other examples the substrate 4 may be formed from materials such as paper.

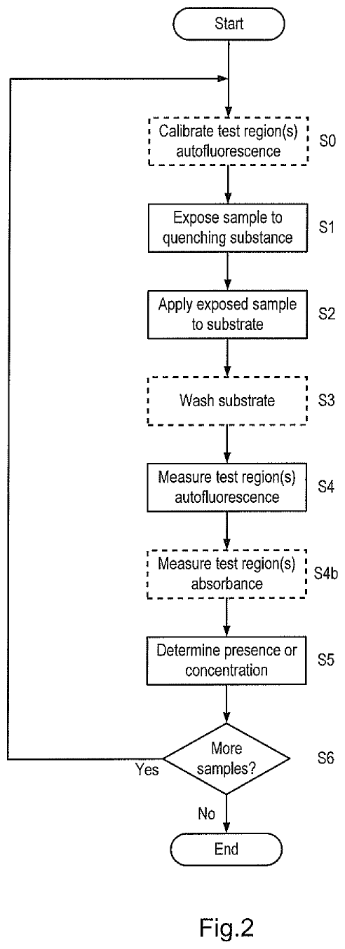

Referring also to FIG. 2, an example of the first fluorescence quenching immunoassay method is explained.

In this example, the substrate 4 comprises at least one test region 7 which has been treated with an immobilised binding reagent 8 for selectively binding an analyte-quenching complex 9.

The liquid sample 2 which is suspected of containing the analyte 1 is exposed to a quenching substance 10 which is capable of selectively binding to the analyte 1 to form the analyte-quenching complex 9 (step S1). The quenching substance 10 includes a quenching label 11 bound to a specific binding reagent 12 which is capable of selectively binding the analyte 1. The quenching label 11 may be any substance, compound or particle which quenches autofluorescence light 5 emission by the substrate 4. For example, the quenching substance 10 may include quenching labels 11 in the form of gold nanoparticles, other metallic nanoparticles, molecular quenchers and so forth. The quenching label 11 may quench the substrate autofluorescence by any suitable mechanism such as, for example, by energy transfer, by electron transfer and/or by absorbing light at one or more wavelengths which excite autofluorescence of the substrate 4 material. The specific binding reagent 12 is generally an antibody, but may be an antigen in some assays.

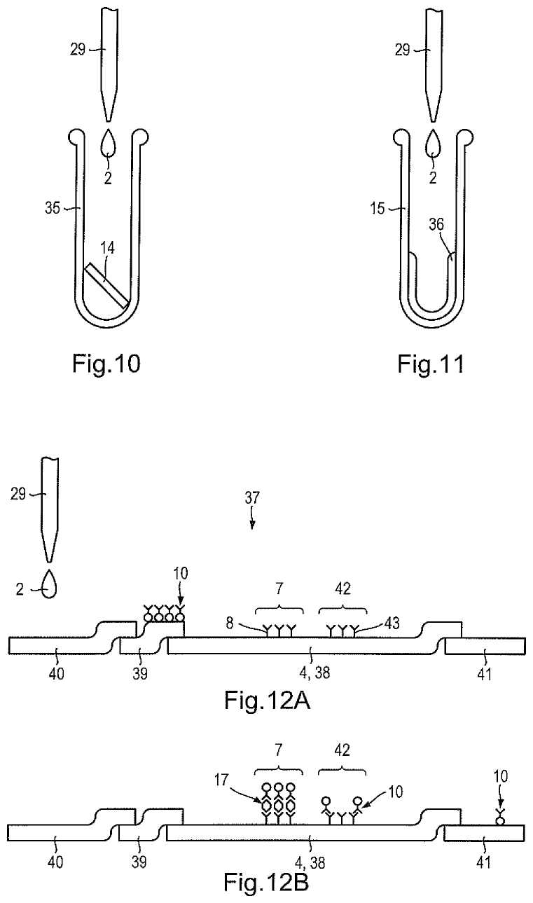

The liquid sample 2 may be exposed to the quenching substance 10 by mixing the liquid sample 2 with a second liquid 13 (FIG. 8) in which the quenching substance 10 has been dissolved or suspended. Alternatively, the liquid sample 2 may be applied to a second substrate 14 (FIG. 10) or a container 15 (FIG. 11) which has been coated or impregnated with the quenching substance 10. In this latter case, the quenching substance 10 may by dissolved by the liquid sample 2.

In the exposed liquid sample 16, the specific binding reagent 12 binds to analyte 1 molecules but does not bind to non-analyte molecules 6. In this way, analyte-quenching complexes 9 are formed which include the analyte 1 bound to the quenching substance 10. After a duration has elapsed following exposure of the liquid sample 2 to the quenching substance 10, the exposed liquid sample 16 is applied to the substrate 4 (step S2).

For example, the exposed liquid sample 16 may be directly applied to the substrate 4 using a pipette or similar apparatus. Alternatively, the exposed liquid sample 16 may be applied to a portion of the substrate 4 away from the test region 7, or to a separate substrate in fluid communication with the substrate 4, and the exposed liquid sample 16 may subsequently be transported to the test region 7 of the sample 4 along a liquid transport path, for example by capillary action. When the exposed liquid sample 16 is applied directly to the substrate 4, the duration between exposure and application may be pre-determined. Alternatively, when the exposed liquid sample 16 is transported to the test region 7, for example by capillary flow, the duration may be determined by the length, porosity, materials and/or other properties of the liquid transport path.

Once the exposed liquid sample 16 has been applied/transported to a test region 7 of the substrate 4, the immobilised binding reagent 8 specifically binds to the analyte-quenching complexes 9 to form an immobilised-quenching-complex 17 which includes the immobilised binding reagent 8, the analyte 1, the specific binding reagent 12 and the quenching label 11. The immobilised binding agent 8 generally takes the form of an antibody, however, in some assays an antigen may be used. The immobilised binding reagent 8 is specific in the sense that the immobilised binding reagent 8 does not bind non-analyte molecules 6, unbound labelling substance 10 and so forth. The immobilised binding agent 8 is immobilised in the sense that it is attached to the substrate 4 within the test region 7, at least sufficiently to prevent removal by the flow of exposed liquid sample 16 over and/or through the substrate 4.

Optionally, the substrate 4 may be washed or rinsed with water or another suitable solution or solvent to ensure that any unbound labelling substance 10 and/or non-analyte molecules 6 are removed prior to measuring the sample 4 autofluorescence signal 3 (step S3).

The autofluorescence signal 3 of the substrate 4 is measured, in particular the autofluorescence signal 3 corresponding to the, or each, test region 7 is measured (step S4). For example, the autofluorescence signal 3 may be measured by exposing the substrate 4 to exciting light 18 and by detecting the resulting autofluorescence light 5 emitted by the substrate 4 using a photodetector. In general, the photodetector should be arranged to avoid or minimise detection of stray and/or scattered exciting light 18. For example, by using a filter which blocks the exciting light 18 or by using a photodetector which is insensitive, or has low sensitivity, to the exciting light 18. The exciting light 18 may be provided by a specially provided light source, or the exciting light 18 may be ambient light. When ambient light is used, the substrate 4 may be illuminated by ambient light through a filter which passes the desired exciting light, for example UV or blue light, whilst attenuating light at wavelengths where the substrate 4 exhibits autofluorescence. The practicality of using ambient light may be limited by the intensity and variability of ambient light, and also by the strength of the autofluorescence response of the substrate 4 to ambient light.

The presence or concentration of the analyte 1 within the, or each, test region 7 is determined in dependence upon a decrease ΔI (also referred to as a quenching) in an autofluorescence signal 3 measured for that test region (step S5). The decrease ΔI may be determined with reference to an initial autofluorescence signal 3 corresponding to the test region 7 prior application of the exposed liquid sample 16. A reference initial autofluorescence signal 3 may be measured during a calibration step prior to commencing the assay (step So). Alternatively, the decrease ΔI may be determined by reference to the autofluorescence signal 3 observed for regions of the sample 4 adjacent to the or each test region 7 and which have not been treated with the immobilised binding reagent 8.

If there are more liquid samples 2 to test, the process is repeated (step S6).

Further features of the fluorescence quenching immunoassay method shall become apparent with reference to the examples described hereinafter.

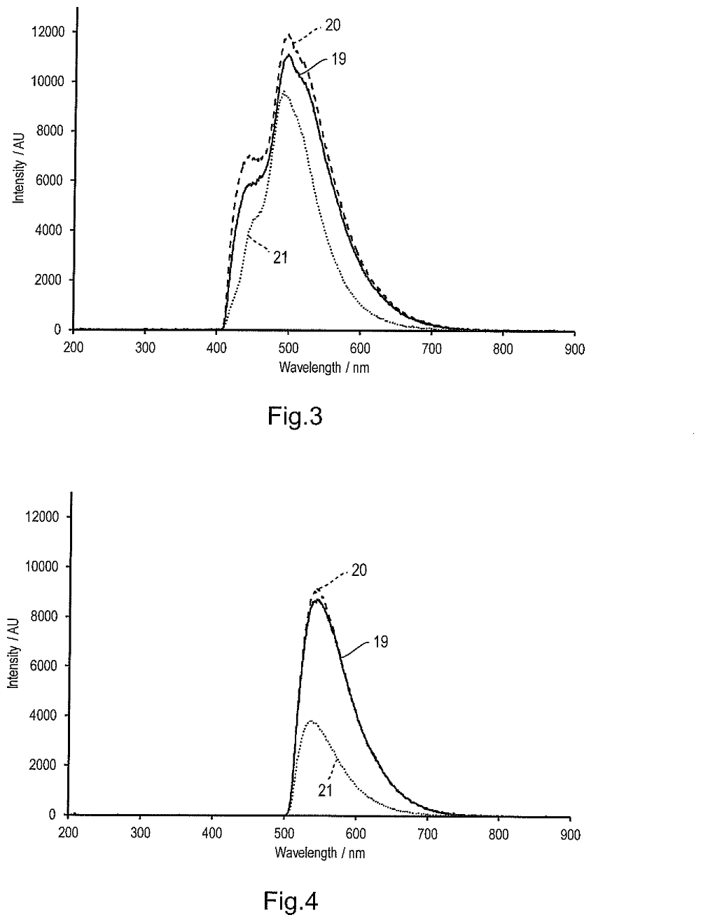

Referring also to FIGS. 3 and 4, autofluorescence measurements for substrates 4 including nitrocellulose fibres are shown for the cases of exciting light 18 at ultra-violet (UV) wavelengths (FIG. 3) or blue wavelengths (FIG. 4). The exciting light 18 at UV wavelengths was between 365 nm to 367 nm, and the exciting light 18 was filtered out from the autofluorescence spectra (FIG. 3) using a long-pass filter with a cut-off wavelength between 405 and 410 nm. The exciting light 18 at blue wavelengths was between 445 nm to 490 nm, and the exciting light 18 was filtered out from the autofluorescence spectra (FIG. 4) using a long-pass filter with a cut-off wavelength at 500 nm.

Referring in particular to FIG. 3, autofluorescence spectra are shown corresponding to a sample of nitrocellulose having no backing 19 (solid line), a sample of nitrocellulose having a clear backing 20 (dashed line) of 100 μm thickness polyester, and a sample of nitrocellulose supported on a glass slide 21 (dotted line). FIG. 4 shows data from the same samples 19, 20, 21, except that each sample 19, 20, 21 was illuminated using exciting light 18 at blue wavelengths. None of the nitrocellulose samples contained or were treated with any fluorophores of phosphorescent materials such as fluorescent or phosphorescent tags, dyes and so forth.

It may be observed from FIGS. 3 and 4 that nitrocellulose has a fluorescent signal even in the absence of fluorescent tags. This phenomenon of many organic materials is generally known as autofluorescence, to distinguish it from fluorescence by specifically added tags/dyes and so forth. Typically, autofluorescence is less intense and occurs across a wider wavelength range compared to fluorescence of specifically added tags/dyes. FIGS. 3 and 4 demonstrate that autofluorescence of nitrocellulose may be excited by exciting light 18 at UV and blue wavelengths. However, some autofluorescence may even be observed using exciting light 18 at red wavelengths.

The comparison between samples without backing 19 and with clear backing 20 demonstrates that the observed autofluorescence signals 3 are dominated by the nitrocellulose itself, rather than any lamination or backing. Both the sample without backing 19 and the sample with clear backing 20 included nitrocellulose layers at least 200 μm thick. The samples 19, 20 with and without backing may also be compared to nitrocellulose on a glass slide 21, which has a fluorescent signal which is lower. The reduced autofluorescence signal from the nitrocellulose on glass slide 21 may result from the nitrocellulose being 12 μm thick, which is significantly thinner than the samples 19, 20 with and without backing. Porous substrates 4 made from nitrocellulose fibres are commonly used in lateral flow immunoassay tests. However, nitrocellulose substrates 4 may be used in other types of assay. Furthermore, many other materials such as, for example, cellulose, lignin, collagen, cotton, silk, paper and so forth may also display autofluorescence which may be utilised for conducting an autofluorescence quenching assay in the same way as nitrocellulose.

The present disclosure may be understood with reference to the problems encountered in assays based on quenching of intentionally added fluorophores such as dyes, tags, proteins and so forth.

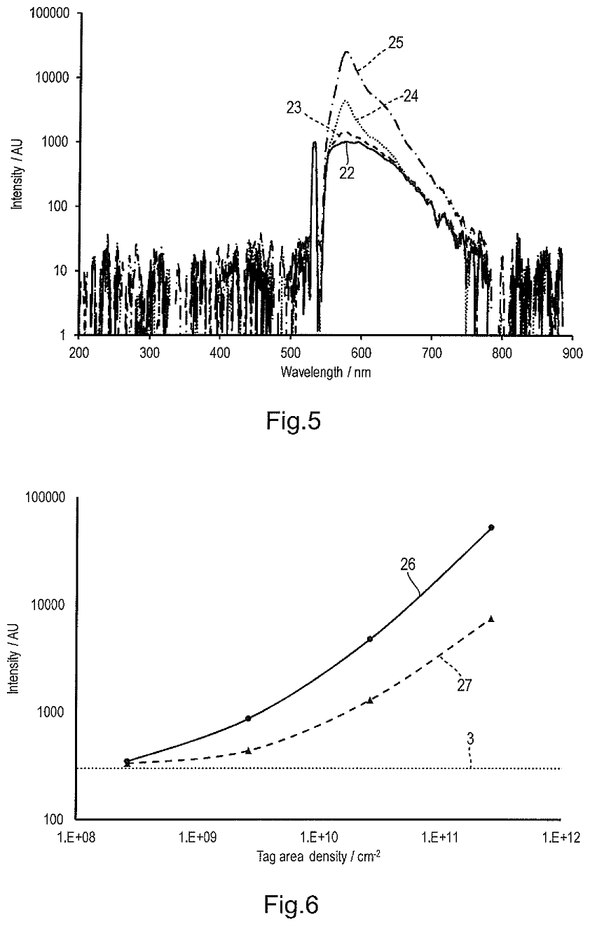

Referring also to FIG. 5, fluorescence spectra are shown for a fluorescent protein R-PE on a substrate 4 made from nitrocellulose.

FIG. 5 includes data from four samples. A first sample 22 was a substrate 4 formed from nitrocellulose fibres (solid line) and untreated with R-PE. A second sample 23 was a substrate 4 formed from nitrocellulose fibres and treated with a solution of 0.0001% R-PE (dashed line). A third sample 24 was a substrate 4 formed from nitrocellulose fibres and treated with a solution of 0.001% R-PE (dotted line). A fourth sample 25 was a substrate 4 formed from nitrocellulose fibres and treated with a solution of 0.01% R-PE (chained line). Measurements were conducted using a green laser to provide excitation light, and a 550 nm long pass filter. It may be observed that although R-PE provides a strong fluorescence response, the autofluorescence signal of the substrate 4 formed of nitrocellulose becomes a limiting factor at low R-PE concentrations.

Referring also to FIG. 6, the peak fluorescence intensity is shown as a function of R-PE tag area density for transmission 26 (solid line) and reflection 27 (dashed line) geometries. The autofluorescence signal 3 is also plotted in FIG. 6.

It may be observed that the autofluorescence signal 3 limits the measurement in both reflection and transmission geometries as the area density of R-PE fluorescent tags is decreased

In a conventional fluorescence quenching based assay the autofluorescence signal 3 is considered unwanted and a number of methods have been employed to minimise it. For example, phosphorescent tags may be used in combination with time-gating to only measure the wanted signal. Fluorescent dyes/tags with a large Stoke's shift may also be used, although there is still likely to be a contribution from autofluorescence, which may still dominate in the low analyte concentration range.

By contrast, instead of viewing autofluorescence as a problem which must be minimised or corrected for, the inventors of the present specification have realised that the autofluorescence signal itself may be quenched as the basis for an immunoassay method. Quenching of the autofluorescence signal of a substrate 4 may well occur in conventional fluorescence assays, but this is either not measured (for example in assays based on time gating or large Stokes shift), or is indistinguishably mixed up with the quenching of a fluorescent tag/dye/marker. By omitting any fluorescent tags and intentionally quenching the substrate 4 autofluorescence, a fluorescence quenching immunoassay method may be made less complex and avoid the need for expensive fluorescent tags/dyes/markers.

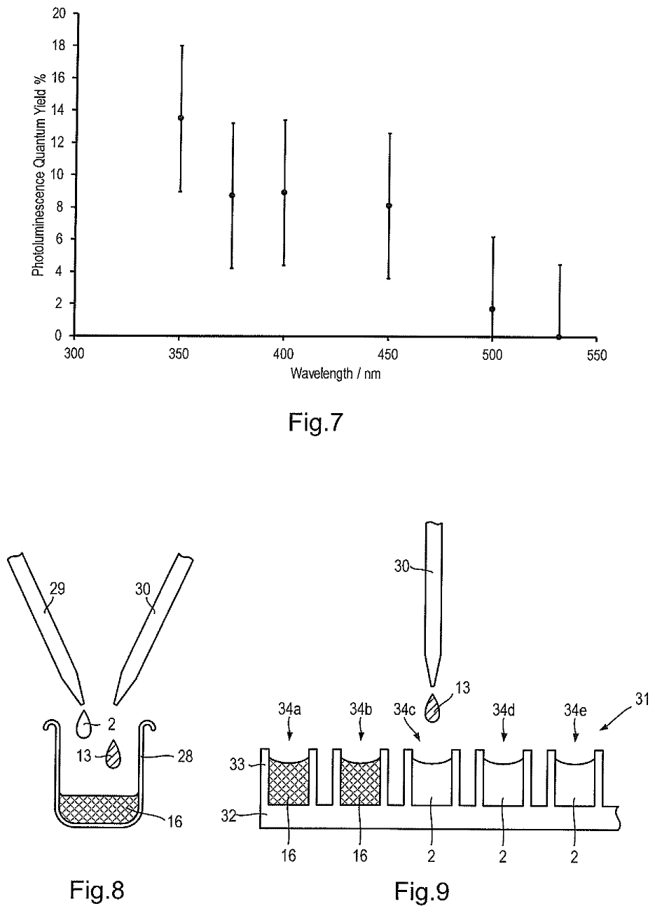

Referring also to FIG. 7, the photoluminescence quantum yield (PLQY) of nitrocellulose (including laminated 100 μm polyester backing) is shown as a function of wavelength.

It may be observed that the PLQY of nitrocellulose is approximately 10% in the UV-blue wavelength region. Thus, even though the PLQY is relatively low, there is expected to be a sufficient autofluorescence signal 3 to enable detection of quenching.

Referring also to FIG. 8, a first method of exposing the liquid sample 2 to the quenching substance 10 is shown.

Liquid sample 2 may be added to a container 28 such as a beaker, test tube, cuvette and so forth. The liquid sample 2 may be already present in the container 28 or may be added using any suitable means such as, for example, a pipette dropper 29 or syringe (not shown) filled with liquid sample 2. Subsequently, a quantity of the second liquid 13 containing the quenching substance 10 may be added to the container 28 using any suitable means such as, for example, a pipette dropper 30 or syringe (not shown) filled with second liquid 13. The liquid sample 2 and the second liquid 13 mix in the container to form the exposed liquid sample 16. The exposed liquid sample 16 may then be transferred/applied to the substrate 4 from the container 28.

Referring also to FIG. 9, a second method of exposing the liquid sample 2 to the quenching substance 10 is shown.

The second method uses an assay plate 31 to enable quick and convenient testing of multiple liquid samples 2. The assay plate 31 includes a transparent base 32. A number of hollow cylinders 33 extend perpendicularly from the transparent base 32 to provide a number of sample wells 34, for example a first sample well 34a, second sample well 34b and so forth. Each sample well 34 may be provided with a different liquid sample 2, or several sample wells 34 may hold the same liquid sample 2 to allow repetition/verification of assay results. For example, the first sample well 34a may hold a first liquid sample 2a, the second sample well 34b may hold a second liquid sample 2b and so forth. The sample wells 34 may extend in one direction. More typically, the sample wells 34 extend in two directions to form an array.

A suitable liquid transfer means such as, for example, a pipette 30 or syringe (not shown) filled with the second liquid 13 may be used to add a quantity of the second liquid 13 to each sample well 34. After a duration has elapsed to allow formation of analyte-quenching complex 9, the exposed liquid samples 16 contained by each sample well 34 may be transferred to respective substrates 4 or test regions 7 of substrates 4 in order to continue the fluorescence quenching immunoassay method.

Referring also to FIG. 10, a third method of exposing the liquid sample 2 to the quenching substance 10 is shown.

A container 35 holds a second substrate 14 which has been treated or coated with the quenching substance 10. The liquid sample 2 is added to the container 35 using suitable liquid transfer means such as, for example, a pipette 29 or syringe (not shown) filled with the liquid sample 2. The liquid sample 2 contacts the second substrate 14 and the quenching substance 10, which is not bound or not strongly bound to the second substrate 14, is released into the liquid sample 2 to form analyte-quenching complexes 9. Subsequently, for example after a predetermined duration, the exposed liquid sample 16 may be transferred/applied to a substrate 4 or a test region 7 of a substrate 4 to continue the fluorescence quenching immunoassay method.

Referring also to FIG. 11, a fourth method of exposing the liquid sample 2 to the quenching substance 10 is shown.

A container 15 has been coated with a thin layer 36 of the quenching substance 10. The liquid sample 2 is added to the container 15 using suitable liquid transfer means such as, for example, a pipette 29 or syringe (not shown) filled with the liquid sample 2. The liquid sample 2 contacts the layer 36, and the quenching substance 10, which is not bound or not strongly bound to the container 15 wall, is released into the liquid sample 2 to form analyte-quenching complexes 9. Subsequently, for example after a predetermined duration, the exposed liquid sample 16 may be transferred/applied to a substrate 4 or a test region 7 of a substrate 4 to continue the fluorescence quenching immunoassay method.

Referring also to FIGS. 12A and 12B, a method of conducting the fluorescence quenching immunoassay method using a lateral flow immunoassay strip/device 37 is shown.

In this example, the substrate 4 takes the form of a porous strip 38 formed from a material which exhibits autofluorescence, for example nitrocellulose. The porous strip 38 includes the, or each, test region 7. The strip/device 37 also includes a porous conjugate pad 39 arranged to permit fluid communication with the porous strip 38. The conjugate pad 39 includes the quenching substance 10. For example the conjugate pad 39 has been treated by application of second liquid 13 followed by drying, or other suitable means. The main requirement is that the quenching substance 10 is not bound, or not strongly bound, to the conjugate pad 39, so that the quenching substance may be taken up by the liquid sample 2 upon contact. The strip/device 37 also includes a porous sample receiving pad 40 arranged to permit fluid communication with the conjugate pad 39, and a porous wick pad 41 arranged to permit fluid communication with the porous strip 38 at the opposite end of the substrate 4 to the conjugate pad 39.

Lateral flow immunoassay strips/devices 37 are a variety of biological testing kit. Lateral flow immunoassay strips/devices 37 may be used to test a liquid sample 2 such as, for example, saliva, blood, urine, drinking water, food/drink products, for the presence of an analyte 1. Examples of lateral flow immunoassay strips/devices 37 include home pregnancy tests, home ovulation tests, tests for other hormones, tests for specific pathogens, tests for specific drugs and tests for impurities/contaminants in drinking water or food/drink products.

In a typical lateral flow immunoassay strip/device 37, the liquid sample 2 is introduced to the sample receiving pad 40, and the liquid sample 2 is then drawn along the lateral flow immunoassay strip/device 37 by capillary action (or “wicking”), towards the wick pad 41. The liquid sample 2 is transported through the conjugate portion 39, which is pre-treated with the quenching substance 10. At least a portion of the quenching substance 10 is taken up by the liquid sample 2, for example by dissolution or in suspension, and analyte 1 present in the liquid sample 2 reacts with the quenching substance 10 to form analyte-quenching complexes 9. By contrast, the specificity of the specific binding reagent 12 (for example an antibody/antigen) means that the quenching substance 10 does not react with non-analyte molecules 6. The bound analyte-quenching complexes 9, and also unreacted quenching substance 10 and any non-analyte molecules 6 continue to propagate along the lateral flow immunoassay strip/device 37 before reaching the test region 7.

The test region 7 is pre-treated with the immobilised binding reagent 8 (e.g. antibody/antigen) which binds the analyte-quenching complexes 9 to form immobilised-quenching-complexes 17, and which does not bind unreacted quenching substance 10 or non-analyte molecules 6. The immobilised-quenching-complexes 17 include the quenching labels 11, which act to quench the autofluorescence of the substrate 4 material within the test region 7. The quenching labels 11 may quench the autofluorescence of the substrate 4 material by any suitable mechanism such as, for example, energy transfer, electron transfer, or even simply by absorbing or scattering exciting light 18 before it can interact with the substrate 4 material. The development of a concentration of quenching labels 11 in the test region 7 may be measured and quantified based on a decrease ΔI in autofluorescence signal 3 (for fixed intensity of exciting light 18) the when compared to the autofluorescence signal 3 of the test region 7 before conducting the assay, or when compared to the autofluorescence signal 3 of the substrate 4 outside the test region 7.

Although an immunoassay has been described in which the concentration of analyte is proportionate to the concentration of quenching labels 11 in the test region 7, the method of conducting a fluorescence quenching immunoassay is equally applicable to other types of immunoassay test.

The fluorescence quenching method may be performed on developed lateral flow immunoassay strips/devices 37, i.e. the liquid sample 2 has been left for a pre-set period to be drawn along the lateral flow immunoassay strip/device 37. Alternatively, fluorescence quenching method may involve obtaining kinetic, i.e. dynamic time resolved measurements which track any decrease in autofluorescence signal 3 over time as a concentration of quenching labels 11 in the test region 7 increases.

Some lateral flow immunoassay strips/devices 37 also include one or more control regions 42. The control region 42 contains second immobilised binding reagent 43. The second immobilised binding reagent 43 takes the form of an antibody or antigen which selectively (or non-selectively) binds any unreacted quenching substance 10 within the control region 42. This may provide verification that the liquid sample 2 passed through the porous strip 38 and/or that there was no error in the release of the quenching substance 10 from the conjugate pad 39. The concentration of unreacted labelling particles 10 in the control region 42 may be determined in the same way based on quenching of the substrate 4 autofluorescence signal 3 within the control region 42. Some unreacted quenching substance 10 may reach the wick pad 41.

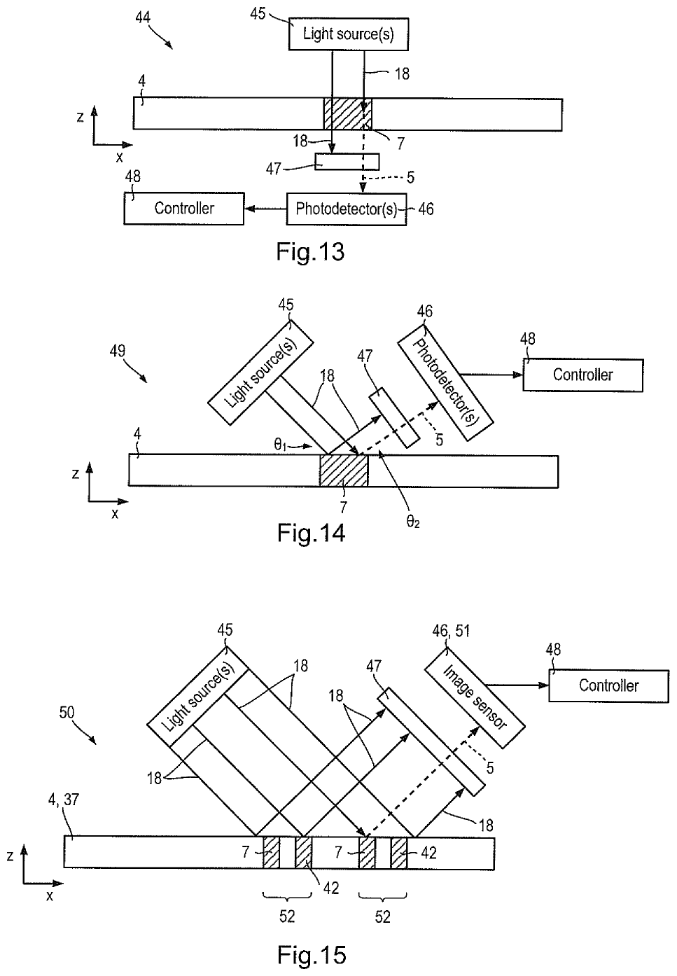

Referring also to FIG. 13, a first fluorescence quenching immunoassay device 44 is shown.

The first device 44 includes one or more excitation light sources 45 spaced apart from one or more photodetectors 46 to define a gap into which a test region 7 of a substrate 4 may be received. The exposed liquid sample 16 is applied/transported to the test region 7 before or after placing the test region 7 between the light source(s) 45 and photodetector(s) 46. Alternatively, the positions of the light source(s) 45 and photodetector(s) 46 may be fixed with respect to the substrate 4, for example, when the first device 44 is a lateral flow immunoassay device 37. The, or each, photodetector 46 is configured to detect the autofluorescence signal 3 from the substrate 4. In the example shown in FIG. 13, the photodetector(s) 46 are configured to detect the autofluorescence signal 3 using a filter 47 which blocks, or at least significantly attenuates, the exciting light 18. Preferably, the filter 47 attenuates the exciting light 18 to 1/10^{th} of the incident intensity or less. Alternatively, the first device 44 may use photodetectors which are inherently insensitive to the wavelength of the exciting light 18.

The output of the photodetector(s) 46 is received by a controller 48 which is configured to determine the presence or concentration of the analyte 1 in dependence upon quenching of, i.e. a decrease ΔI in the autofluorescence signal 3 of the substrate 4 in response to an accumulation of quenching labels 11 within a test region 7 of the substrate 4. Since each quenching label 11 is bound to one analyte 1, the concentration of the quenching labels 11 is substantially the same as the concentration of analyte 1 (some unbound quenching labels 11 may be in transit through the test region 7 at the measurement time and/or be deposited without binding as the exposed liquid sample 16 dries).

Although in this example the exciting light 18 is provided by excitation light sources 45, in alternative examples the exciting light 18 may be ambient light such as natural daylight. When ambient light provides the exciting light 18, the substrate 4 may be exposed to the ambient light through a second filter (not shown) which blocks or substantially attenuates light within the wavelength range of the autofluorescence light 5. The practicality of using ambient light may be limited by the intensity and variability of ambient light, and by the strength of the autofluorescence response of the substrate 4 to ambient light.

Additional optical components may be included in the optical path between the lights source(s) 45 and the photodetector(s) 46, including, but not limited to, slits or other apertures, diffusers, lenses, additional filters, beamsplitters, and so forth.

The first device 44 is configured with the light source(s) 45 and photodetector(s) 46 on opposite sides/faces of the substrate 4, analogous to a transmission geometry for absorbance measurements. However, other configurations may also be used.

For example, referring also to FIG. 14, a second fluorescence quenching immunoassay device 49 is shown.

The second device 49 is similar to the first device 44, except that the second device 49 is configured with the light source(s) 45 and photodetector(s) 46 facing the same side/face of the substrate 4, analogous to a reflection geometry for absorbance measurements.

In this way, the light source(s) 45 of the second device 49 are arranged to illuminate a test region 7 of a sample 4 at first angle θ₁, and the photodetector(s) 46 are arranged to receive light reflected and/or emitted from the substrate 4. Light reflected and/or emitted from the substrate 4 will, in general, be scattered into a wide range of different angles. Consequently, the photodetector(s) 46 may be oriented so as to receive light reflected and/or emitted from the substrate 4 at a second angle θ₂, which does not need to be equal to the first angle θ₁. In some examples, the first and second angles θ₁, θ₂ may be equal. In some examples, the light source(s) 45 and photodetector(s) 45 may be arranged in a confocal configuration. Light reflected and/or emitted from the substrate 4 may originate from a surface of the substrate 4 or from a depth within the substrate 4.

Additional optical components may be included in the optical path between the light source(s) 45 and the photodetector(s) 46, including, but not limited to, slits or other apertures, diffusers, lenses, additional filters, beamsplitters, and so forth.

Referring also to FIG. 15, a third fluorescence quenching immunoassay device 50 is shown.

The third device 50 includes a number of photodetectors 46 arranged in an array to form an image sensor 51. For example, the image sensor 51 may form part of a camera. The image sensor 51 may be arranged to image all of, or a portion of, one or more test regions 7 of the substrate 4. For example, when a substrate 4, for example a lateral flow test strip device 37, is received into the third device 50, the image sensor 51 may be arranged to image one or more test regions 7 and the surrounding areas of the substrate 4 material. For example, a substrate 4 in the form of a lateral flow test strip 37 may include one or more pairs 52, each pair 52 including a test region 7 and a control region 42, and the image sensor 51 may be arranged to image the one or more pairs 52 at the same time. The image sensor 51 is arranged to image the substrate 4 through the filter 47, so that the image obtained is based on autofluorescence light 5 transmitted by the filter 47.

The optical path of the third device 50 may be arranged similarly to the first device 44, with the light source(s) 45 and image sensor 51 arranged on opposite sides/faces of the substrate 4. Alternatively, as shown in FIG. 15, the optical path of the third device 50 may be arranged similarly to the second device 49, with the light source(s) 45 and image sensor 51 both arranged to face towards the same side/face of the substrate 4.

Additional optical components may be included in the optical path between the lights source(s) 45 and the photodetector(s) 46, including, but not limited to, slits or other apertures, diffusers, lenses, additional filters, beamsplitters, and so forth.

Combined Auto-Fluorescence Quenching and Absorbance Measurements

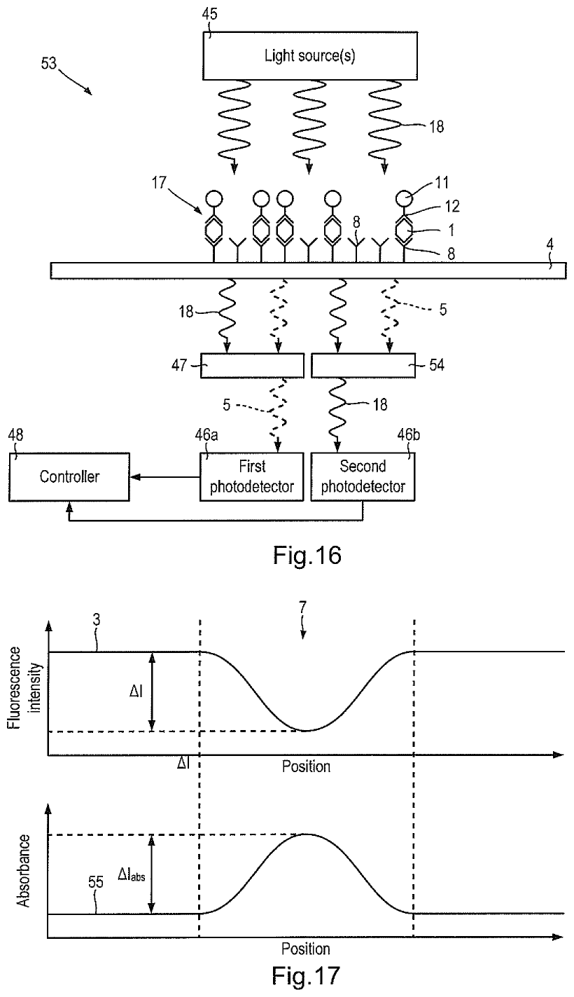

Referring also to FIG. 16, a combined fluorescence quenching and absorbance immunoassay device 53 is shown.

The combined device 53 is similar to the first device 44, except that the combined device 53 includes at least one first photodetector 46a and at least one second photodetector 46b. As described hereinbefore in relation to the first, second and third devices 44, 49, 50, the light source(s) 45 emit light within a range of wavelengths Δλₑₓ which excite the substrate 4 autofluorescence, i.e. a range of one or more excitation wavelengths Δλₑₓ. As described hereinbefore, the quenching labels 11 are selected so as to quench the autofluorescence signal 3 of the substrate 4. However, for use with the combined device 53, the quenching labels 11 and/or the excitation wavelengths Δλₑₓ are also selected such that the quenching label 11 exhibits a measureable absorbance with an absorption wavelength range Δλ_{obs} which overlaps the range of excitation wavelengths Δλₑₓ.

Referring also to FIG. 17, quenching ΔI of the autofluorescence signal 3 and an increase ΔI_{obs} in an absorbance signal 55 are schematically illustrated as a function of position relative to a test region 7.

Each first photodetector 46a is configured to detect autofluorescence light 5 emitted from the substrate, so as to enable measurement of the quenching ΔI of the autofluorescence signal 3 arising from the concentration of quenching labels 11. By contrast, each second photodetector 46b is configured to detect exciting light 18 transmitted through the substrate 4, so as to enable measurement of an increase ΔI_{obs} in the absorbance signal 55 arising from the concentration of quenching labels 1. Preferably, the first and second photodetectors 46a, 46b are closely spaced. For example, the first and second photodetectors 46a, 46b are interdigitated.

For example, in the example shown in FIG. 16, a first photodetector 46a is configured to detect autofluorescence light 5 using the filter 47, which rejects, or at least substantially attenuates, exciting light 18 transmitted through the substrate 4. Similarly, a second photodetector 46b is configured to detect exciting light 18 using a second filter 54. The second filter 54 is configured to transmit the exciting light 18 with no, or minimal, attenuation whilst rejecting, or at least substantially attenuating, autofluorescence light 5 emitted from the substrate 4. When the first and second photodetectors 46a, 46b are interdigitated, the filters 47, 54 are correspondingly interdigitated.

Alternatively the, or each, first photodetector 46a may be inherently sensitive to the autofluorescence light 5 and inherently insensitive to the exciting light 18, and each second photodetector 46b may be inherently sensitive to the exciting light 18 and inherently insensitive to the autofluorescence light 5. Such inherent sensitivity may be provided by using photodetectors 46a, 46b which include different optically active materials, which include microcavities tuned to the autofluorescence light 5 or exciting light 18 respectively, and so forth.

The controller 48 receives signals from the first photodetector(s) 46a and determines the quenching ΔI of the autofluorescence signal 3 as described hereinbefore, namely by reference to an initial autofluorescence signal 3 or by reference to autofluorescence signals 3 obtained from portions of the substrate 4 outside the test region 7.

Autofluorescence signals 3 from other portions of the substrate 4 may be obtained using additional photodetectors 46a which lie outside a test region 7, or by translation of the substrate 4 and/or photodetectors 46a.

Referring again to FIG. 2, the controller 48 also determines the increase ΔI_{obs} of the absorbance signal 55 by comparing signals received from second photodetector(s) 46b by reference to an initial absorbance of the substrate 4 obtained prior to commencing the assay (step S4b). Alternatively, the increase ΔI_{obs} of the absorbance signal 55 may be determined by comparing signals received from second photodetector(s) 46b by reference to absorbance signals 55 obtained from portions of the substrate 4 outside the test region 7. Absorbance signals 55 from other portions of the substrate 4 may be obtained using additional photodetectors 46a which lie outside a test region 7, or by translation of the substrate 4 and/or photodetectors 46a.

For each test region 7 measured, the controller is configured to determine the presence or concentration of the analyte 1 in dependence upon a combination of the change ΔI in the autofluorescence signal 3 from the test region 7, and the change ΔI_{obs} in absorbance signal 55 of the test region 7.

In this way, by utilising both quenching of the autofluorescence signal 3 and also absorbance of the exciting light 18 by the quenching labels 11, the concentration of analyte 1 may be determined with improved accuracy and sensitivity. For example, the concentration of analyte 1 may be determined as the mean average of a first concentration value determined based on the autofluorescence signal 3 and a second concentration value determined based on the absorbance signal 55.

Furthermore, by utilising both quenching of the autofluorescence signal 3 and also absorbance of the exciting light 18 by the quenching labels 11, an estimate of the quality and reliability of the determined analyte concentration may be obtained. For example, a quality estimate may be obtained based on the difference between a first sa concentration value determined based on the autofluorescence signal 3 and a second concentration value determined based on the absorbance signal 55. A large deviation may indicate a defective or unreliable assay result.

The combined device 53 has been described with reference to an example in which, similar to the first device 4, the light source(s) 45 and photodetectors 46a, 46b are arranged on opposite sides/faces of the substrate, so that the absorbance signal 55 is obtained in a transmission geometry. However, alternative combined devices (not shown) may be configured similarly to the second device 49 so that the absorbance signal 55 is obtained in a reflection geometry instead. Still other alternative combined devices (not shown) may be configured to employ image sensors 51 in a similar way to the third device 50.

Lateral Flow Quenching Assay Device

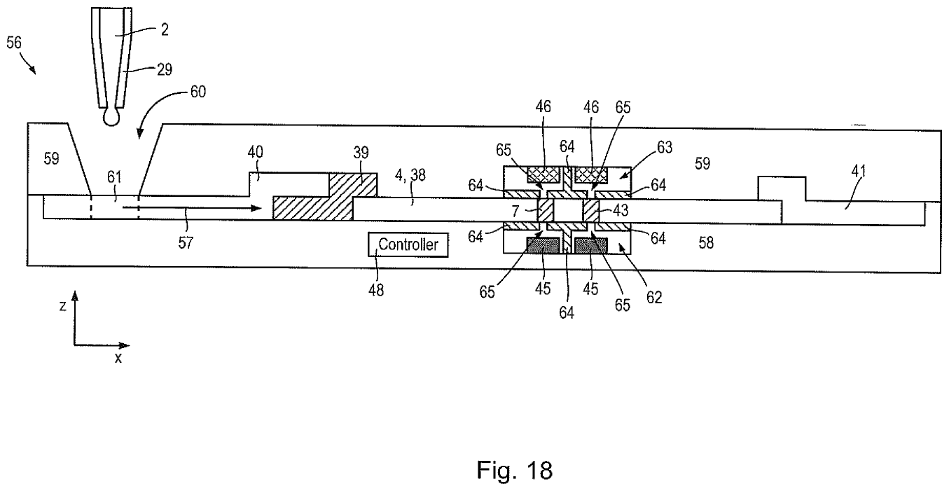

Referring also to FIG. 18, the first fluorescence quenching immunoassay device 44 may be integrated into a self-contained, single use lateral flow testing device 56.

The lateral flow testing device 56 includes a porous sample receiving pad 40 for receiving the liquid sample 2. The lateral flow testing device 56 also includes a porous conjugate pad 39 arranged to permit fluid communication with the sample receiving pad 40. The conjugate pad 39 contains the quenching substance 10 for selectively binding to the analyte 1 to form the analyte-quenching complex 9. The lateral flow testing device 56 also includes the substrate 4 in the form of a porous strip/pad 38 arranged to permit fluid communication with the conjugate pad 39. The porous strip 38 includes at least one test region 7 treated with the immobilised binding reagent 8 for selectively binding analyte-quenching complexes 9. The substrate 4 in the form of the porous strip is formed of a material which exhibits autofluorescence. The lateral flow testing device 56 also includes the wick pad 41 arranged to permit fluid communication with the porous strip 38.

The lateral flow testing device 56 also includes at least one photodetector 46, each photodetector 46 being configured to detect an autofluorescence signal 3 of a corresponding test region 7 or control region 42. The lateral flow testing device 56 also includes a controller 48 configured to determine the presence or concentration of the analyte 1 in dependence upon a decrease ΔI in the autofluorescence signal 3 or signals 3 corresponding to the, or each, test region 7 or control region 42. The liquid sample 2 applied to the sample receiving pad 40 is drawn towards the wick pad 41, in a flow direction 57 through the conjugate pad 39 and the porous strip 38, by a wicking or capillary flow mechanism. Optionally, the lateral flow testing device 56 may also include one or more light sources 45 configured to excite autofluorescence of the porous strip 4 material.

The quenching substance 10 may include a quenching label 11 which quenches the autofluorescence of the porous strip 38 material. The quenching label 11 may be bound to an antibody or an antigen. The immobilised binding agent 8 may include an antibody or an antigen. The quenching label 11 may quench the autofluorescence of the porous strip 38 material by energy transfer, by electron transfer and/or by absorbing light at one or more excitation wavelengths of the porous strip 38 material. The quenching labels 11 may take the form of gold nanoparticles.

The single use lateral flow testing device 56 is packaged such that the sample receiving pad 40, conjugate pad 39, porous strip 38 and wick pad 41 are received into a base 58. A lid 59 is attached to the base 58 to secure the sample receiving pad 40, conjugate pad 39, porous strip 38 and wick pad 41 and to cover parts which do not require exposure for purposes of receiving liquid sample 2, illumination, viewing, measurements and so forth. The lid 59 includes a sample receiving window 60 which exposes part of the sample receiving pad 40 to define the liquid sample receiving region 61. The lid and base 58, 59 are made from a polymer such as, for example, polycarbonate, polystyrene, polypropylene or similar materials.

In the example shown in FIG. 18, the base 58 includes a recess 62 into which a pair of light sources 45 are received. Each light source 45 may be configured as described hereinbefore. The light sources 45 may take the form of light emitting diodes (LEDs), and preferably organic light emitting diodes (OLEDs). The lid 59 includes a recess 63 into which a pair of photodetectors 46 are received. When filters 47 are used to block exciting light 18, the filter 47 are preferably laminated to, or incorporated within, the corresponding photodetectors 46. The photodetectors 46 may take the form of photodiodes, preferably organic photodiodes (OPDs). One pair of a light source 45 and a photodetector 46 are arranged on opposite sides of a test region 7 of the porous strip 38. The second pair of a light source 45 and a photodetector 46 are arranged on opposite sides of a control region 42 of the porous strip 38.

Slit members 64 separate the light sources 45 from the porous strip 38 to define narrow slits 65 with widths in the range between 100 μm and 1.5 mm inclusive, more preferably between 300 μm to 500 μm inclusive. The slit members 64 define slits 65 which extend transversely across the width of the porous strip 38. For example, if the porous strip 38 extends in a first direction x and has a thickness in a third direction z, then the slits 65 extend in a second direction y. Further slit members 64 define slits 65 which separate the photodetectors 46 from the porous strip 38. The slits 65 may be covered by a thin layer of transparent material (not shown) to prevent moisture entering into the recesses 62, 63. Material may be considered to be transparent to a particular wavelength A if it transmits more than 75%, more than 85%, more than 90% or more than 95% of the light at that wavelength A. A diffuser (not shown) may optionally be included between each light source 45 and the corresponding slit 65. As an alternative to having the filters 47 laminated to, or incorporated within, the photodetectors 46, the filters 47 may be provided in place of the thin layer of transparent material (not shown) to prevent moisture entering into the recess 63.

A liquid sample 2 is introduced to the sample receiving pad 40 through the sample receiving window 60 using, for example, a dropper 29, syringe (not shown) or similar implement. The liquid sample 2 is transported in the flow direction 57 towards the wick pad 41 by a capillary, or wicking, action of the porosity of the sample receiving pad 40, conjugate pad 39, porous strip 38 and wick pad 41. The sample receiving pad 40 is typically made from fibrous cellulose filter material.

As described hereinbefore, the conjugate pad 39 is pre-treated with the quenching substance 10. The conjugate pad 39 is typically made from fibrous glass, cellulose or surface modified polyester materials.

As the flow front of liquid sample 2, 16 moves into the porous strip 38 providing the substrate 4, analyte-quenching complexes 9 and unbound quenching substance 10 are carried along towards the wick pad 41. The porous strip 38 includes one or more test regions 7 and control regions 42 which are monitored by corresponding light source 45 and photodetector 46 pairs. Each test region 7 is pre-treated with the immobilised binding reagent 8 which specifically binds the analyte-quenching complexes 9 and which does not bind the unreacted quenching substance 10 or non-analyte molecules 6. As the analyte-quenching complexes 9 are bound in a test region 7, the concentration of the quenching labels 11 in the test region 7 increases. The concentration increase may be monitored by measuring the decrease in autofluorescence signal 3. The autofluorescence signal 3 of the test region 7 may be measured once a set duration has expired since the liquid sample 2 was added. Alternatively, the absorbance of the test region 7 may be measured continuously, or at regular intervals, as the assay is developed. To provide distinction between a negative test and a test which has simply not functioned correctly, a control region 42 is often provided between in addition to the test region 7. The control region 42 is pre-treated with the second immobilised binding reagent 43 which specifically or non-specifically binds unbound quenching substance 10. In this way, if the lateral flow testing device 56 has functioned correctly and the liquid sample 2 has passed through the conjugate pad 39 and the test region 7, the control region 42 will exhibit a decrease in autofluorescence signal 3. The autofluorescence signal 3 of the control region 42 may be measured by the second pair of a light source 45 and a photodetector 46 in the same way as for the test region 7. The porous strip 38 is typically made from fibrous nitrocellulose, or any other suitable fibrous material which exhibits autofluorescence.

Although an immunoassay has been described in which the concentration of analyte is proportionate to the concentration of quenching labels 11 in the test region 7, the method of conducting a fluorescence quenching immunoassay is equally applicable to other types of immunoassay test.

The wick pad 41 soaks up liquid sample 2 which has passed through the porous strip 38, and helps to maintain through-flow of the liquid sample 2. The wick pad 41 is typically made from fibrous cellulose filter material.

Modifications

It will be appreciated that many modifications may be made to the embodiments hereinbefore described. Such modifications may involve equivalent and other features which are already known in the design, manufacture and use of fluorescence quenching immunoassay methods and/or analytical test devices and which may be used instead of or in addition to features already described herein. Features of one embodiment may be replaced or supplemented by features of another embodiment.

An example of the first fluorescence quenching immunoassay device 44 integrated into a self-contained, single use lateral flow testing device 56 is shown in FIG. 18. Alternatively, either of the second or third fluorescence quenching immunoassay devices, 49, 50 may be integrated into a self-contained, single use lateral flow testing device (not shown) in a similar way to the self-contained lateral flow testing device 56.

Other examples of single use lateral flow testing devices (not shown) may include an integrated combined fluorescence quenching and absorbance immunoassay device 53. For example, the example shown in FIG. 18 may be modified to replace the photodetectors 46 with first and second interdigitated photodetectors 46a, 46b. Such examples may be configured to obtain the absorbance measurements in a transmission geometry (similar to the first device 44 and the combined device 53), in a reflection geometry (similar to the second device 49), or using an image sensor 51 in either transmission or reflection geometries (similar to the third device 50).

Although claims have been formulated in this application to particular combinations of features, it should be understood that the scope of the disclosure also includes any novel features or any novel combination of features disclosed herein either explicitly or implicitly or any generalization thereof. The applicant hereby gives notice that new claims may be formulated to such features and/or combinations of such features during the prosecution of the present application or of any further application derived therefrom.

1. A fluorescence quenching immunoassay method, comprising determining the presence or concentration of an analyte in a liquid sample in dependence upon quenching of an autofluorescence signal of a substrate.

2. A method according to claim 1, wherein the substrate is formed from a material which is inherently fluorescent.

3. A method according to claim 1, wherein the substrate does not comprise any added fluorophores or phosphorescing substances.

4. (canceled)

5. A method according to claim 1, wherein the substrate is formed from fibres comprising one or more of cellulose, nitrocellulose, lignin, collagen, cotton and silk.

6. A method according to claim 1, wherein the method is conducted using a lateral flow immunoassay device.

7. A method according to claim 1, further comprising determining the presence or concentration of the analyte in dependence upon a combination of quenching of the autofluorescence signal of the substrate and an absorbance of a quenching substance which causes quenching of the substrate autofluorescence signal.

8. A method according to claim 1, wherein the substrate comprises at least one test region treated with an immobilised binding reagent for selectively binding an analyte-quenching complex, the method comprising:
exposing the liquid sample to a quenching substance for selectively binding to the analyte to form an analyte-quenching complex;
applying the exposed liquid sample to the substrate;
determining, for each test region, the presence or concentration of the analyte in dependence upon a decrease in a corresponding autofluorescence signal.

9. A method according to claim 8, wherein the quenching substance comprises gold nanoparticles.

10. A method according to claim 8, wherein the substrate autofluorescence is excited by one or more excitation wavelengths, and wherein the quenching substance absorbs within a first wavelength range which overlaps one or more excitation wavelengths;
wherein the presence or concentration of the analyte is determined, for each test region, in dependence upon a combination of:
the change in the autofluorescence signal from the test region in response to illumination with light of the first wavelength range; and
a change in absorbance of the test region within the first wavelength range.

11. A method according to claim 8, wherein the method is conducted using a lateral flow immunoassay device comprising:
a substrate in the form of a porous strip, the porous strip comprising the at least one test region; and
a porous conjugate pad arranged to permit fluid communication with the porous strip, the conjugate pad comprising the quenching substance.

12-13. (canceled)

14. A fluorescence quenching immunoassay device configured to determine the presence or concentration of an analyte in a liquid sample, the device comprising;
a substrate which exhibits autofluorescence;
one or more photodetectors configured to detect an autofluorescence signal of the substrate; and
a controller configured to determine the presence or concentration of the analyte in dependence upon quenching of the autofluorescence signal of the substrate.

15. A device according to claim 14, wherein the substrate is formed from a material which is inherently fluorescent.

16. A device according to claim 14, wherein the substrate does not comprise any added fluorophores or phosphorescing substances.

17. (canceled)

18. A device according to claim 14, wherein the substrate is formed from fibres comprising one or more of cellulose, nitrocellulose, lignin, collagen, cotton and silk.

19. A device according to claim 14, wherein the device is a lateral flow immunoassay device.

20. A device according to claim 14, wherein the one or more photodetectors comprise:
at least one first photodetector configured to detect an autofluorescence signal of the substrate; and
at least one second photodetector configured to detect an absorbance of a quenching substance which causes quenching of the substrate autofluorescence signal;
wherein the controller is further configured to determine the presence or concentration of the analyte in dependence upon a combination of quenching of the autofluorescence signal of the substrate and the absorbance of the quenching substance.

21. A device according to claim 14, the device comprising
a porous sample receiving pad for receiving the liquid sample;
a porous conjugate pad arranged to permit fluid communication with the sample receiving pad, the conjugate pad comprising a quenching substance for selectively binding to the analyte to form an analyte-quenching complex;
the substrate in the form of a porous test pad arranged to permit fluid communication with the conjugate pad, the test pad comprising at least one test region treated with an immobilised binding agent for selectively binding analyte-quenching complexes, wherein the test pad is formed of a material which exhibits autofluorescence;
a wick pad arranged to permit fluid communication with the test pad;
wherein each photodetector is configured to detect an autofluorescence signal of a corresponding test region;
wherein the controller is configured to determine, for each test region, the presence or concentration of the analyte in dependence upon a decrease in a corresponding autofluorescence signal;
wherein the liquid sample applied to the sample receiving pad will be drawn towards the wick pad via the conjugate pad and the test pad by a wicking mechanism.

22. A device according to claim 14, further comprising one or more light emitters configured to excite autofluorescence of the substrate.

23. A device according to claim 21, wherein the quenching substance comprises gold nanoparticles.

24. A device according to claim 21, wherein the one or more light detectors comprise at least first and second photodetectors, wherein the substrate autofluorescence is excited by one or more excitation wavelengths, and wherein the quenching substance absorbs within a first wavelength range which overlaps one or more excitation wavelengths;
wherein the controller is configured to determine, for each test region, the presence or concentration of the analyte in dependence upon a combination of:
the change in the autofluorescence signal from each test region in response to illumination with light of the first wavelength range, measured using the first photodetector; and
a change in absorbance of the test region within the first wavelength range, measured using the second photodetector.

25. (canceled)
